(19) United States
(12) Patent Application Publication
(10) Pub. No.: US 2006/0183157 A1
(43) Pub. Date: Aug. 17, 2006

(54) Methods and devices for characterizing macromolecular complexes using isotope labeling techniques

(76) Inventors: David Cowburn, Westfield, NJ (US); Kaushik Dutta, New York, NY (US); Fabien Ferrage, New York, NY (US); Alexander Shekhtman, Albany, NY (US)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/350,176

(22) Filed: Feb. 7, 2006

Related U.S. Application Data
(60) Provisional application No. 60/650,725, filed on Feb. 7, 2005.

(51) Int. Cl. G01N 33/53; G06F 19/00

(52) U.S. Cl. 435/7.1; 702/19

(57) ABSTRACT

A method for characterizing interactions in macromolecular complexes, such as protein-protein or protein-ligand complexes, by selective isotopic labeling of the target molecule to reduce the ¹H density in a selected spectral region; by irradiating the target; and by monitoring the polarization using filtered nuclear Overhauser spectroscopy (NOESY) and/or by performing selective saturation transfer experiments to determine the docking potential of the macromolecular complex.

This application claims benefit of U.S. Ser. No. 60/650,725, filed Feb. 7, 2005, which is incorporated herein by reference in its entirety.

Throughout this application, various publications are referenced, and disclosures of these publications are hereby incorporated in their entireties by reference into this application to more fully describe the state of the art to which this invention pertains.

### BACKGROUND OF THE INVENTION

The knowledge of how a specific protein or molecule (“target”) interacts with any binding molecule (“ligand”) can provide rational design of agonistic and antagonistic molecules for pharmaceutical, agricultural, and other industrial uses.

Magnetic Resonance Spectroscopy (NMR) has been very efficient in determining the structure and dynamics of biomolecules, including proteins. NMR can detect non-covalent interactions between a target and a ligand by detecting changes in spectra. However, the power of this method is seriously limited by problems of large numbers of signals and the complexity of their resolution and identification.

Nuclear Magnetic Resonance (NMR)

Structural determination of interfaces is an ongoing challenge in biological NMR. Many methods employing differential labeling have been developed and successfully applied to protein-protein and protein-nucleic acids interfaces. [1, 2] A major limitation of most methods is the measurement of intermolecular or interdomain nuclear Overhauser effects (nOes). Due to low complex concentration or significant interdomain motion, these effects are small. Moreover, the use of such methods in high molecular mass systems is challenging because of fast proton relaxation. Saturation transfer methods [2] have proven to be efficient but the information provided is qualitative and limited to nitrogen-bound protons. Monitoring chemical shift changes, upon titration of one partner with respect to another, is useful but gives results which are difficult to use as structural constraints and sometimes ambiguous. [1] To solve the structure of the complex, it is also desirable to provide some form of docking potential based on distances for docking procedures such as High Ambiguity Driven Protein-protein Docking based on Biochemical and/or Biophysical Information (HADDOCK).[4]

### SUMMARY OF THE INVENTION

In accordance with these and other objects of the invention, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

It is an object of the present invention to provide a method for mapping the surface interactions of complexes such as protein-protein complexes by selective isotopic labeling of the target, which reduces the ¹H density in a selected spectral region. Selective irradiation in this region affects directly only the ligand ¹H density, and indirectly through the binding surface, the target molecule.

In an embodiment, the complex comprises a protein with ligands or other protein, other intra-protein domains or synthetic molecules. In another embodiment, the biomolecules are each, independently, a protein, a nucleic acid, a carbohydrate, a natural or synthetic ligand or a lipid. In a further embodiment, the molecules are copolymers.

It is another object of the present invention to provide a method for identifying the specific NMR properties of a spin dilute protein's surface for a ¹H spin dense ligand.

In an embodiment of the present invention, the surface interacting with the ubiquitin interaction motif is identified using modified REDPRO labeling [5] of ubiquitin. In alternative embodiments, interactions of small drug-like molecules with proteins can be characterized; intra-domain interactions in a single chain protein can be characterized with additional use of segmental isotopic labeling [9, 10]; in vivo spectroscopy may be used of cultures over-expressing their target to study the target's interactions with ligands; in vitro conditional cell free expression of a ligand in presence of pre-labeled target, or of target in presence of ligand may be used; application with pooled ligands, or multiple ligands are practical.

This invention provides a method for determining the structure or interfacial dynamics of biomolecular complexes, comprising the steps of: (a) providing one or more low proton density target molecule; (b) providing one or more protonated partner molecule that interacts with the target molecule; (c) labeling the target molecule using reduced proton labeling; (d) irridiating the target and partner molecule using ¹H{¹³C} or ¹H{¹⁵N} Heteronuclear Single Quantum Coherence (HSQC)-edited filtered nuclear Overhauser spectroscopy (nOesy) or similar nOesy experiments; (e) recording the polarization of the target molecule to obtain spectral data of the target molecule; and (f) evaluating the spectral data to obtain the structure or interfacial dynamics of the target and partner molecule.

### DETAILED DESCRIPTION OF THE FIGURES

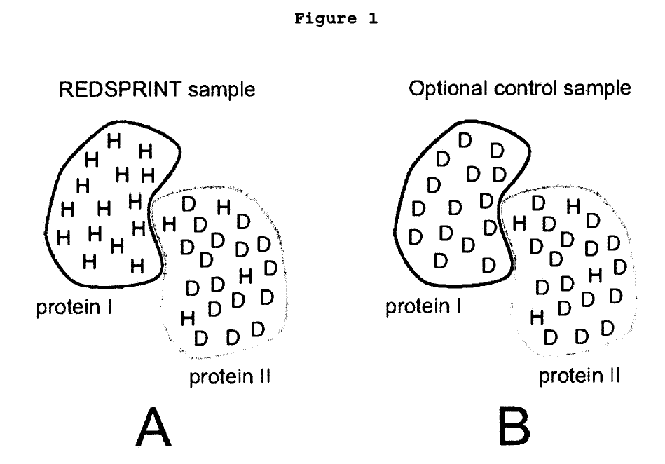

FIG. 1 (A) shows the ataxin 3 UIM (AUIM) sample prepared in a minimal medium with natural abundance isotopes. (B) Control: AUIM was grown in a D₂O minimal medium. Both samples are prepared in a buffered D2O:H2O (80:20) solution.

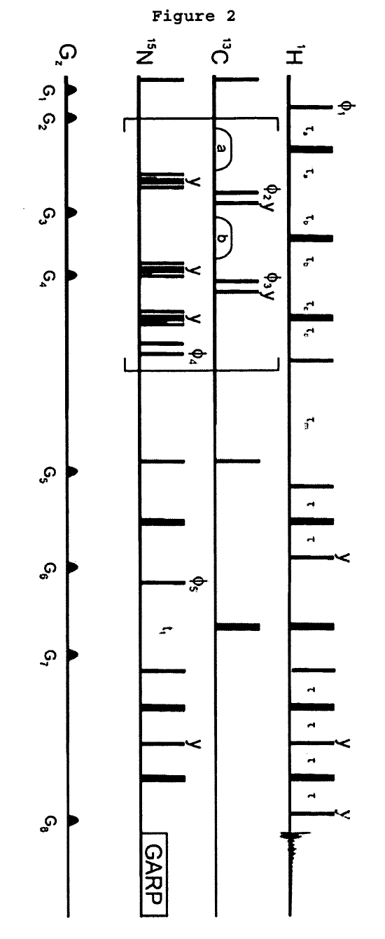

FIG. 2 shows the pulse sequence for the ¹H{¹⁵N} HSQC-edited filtered NOESY experiment. In this adaptation of the filtered NOESY sequence presented in reference [7], only variations will be discussed. The major adaptation is the detection edited by a ¹H{¹⁵N} HSQC. To be adapted for a cold probe, the nitrogen filter is slightly different; the delay τ_{c} was set so that the effective evolution under the 15N-1H scalar coupling is ½ JNH. Sensitivity enhancement was performed with a PEP scheme. Gradients are set so that G2+G4=G3 and G7/G8=9.9. The phase cycle is: φ1=8 {x,x,−x,−x}; φ2=4{4{y}, 4{−y}}; φ3=2{8{y}, 8{−y}}; φ4=16{x}, 16{−x}; φ5=16{x, −x}; φ_{acq}=8{x,−x,−x,x}. The phase cycle of a 90° pulse on the proton channel prior to the τₘ period is necessary to prevent the observation of any steady state during the nOe mixing time. The filter between brackets can be deleted to obtain the intrinsic sensitivity of each signal.

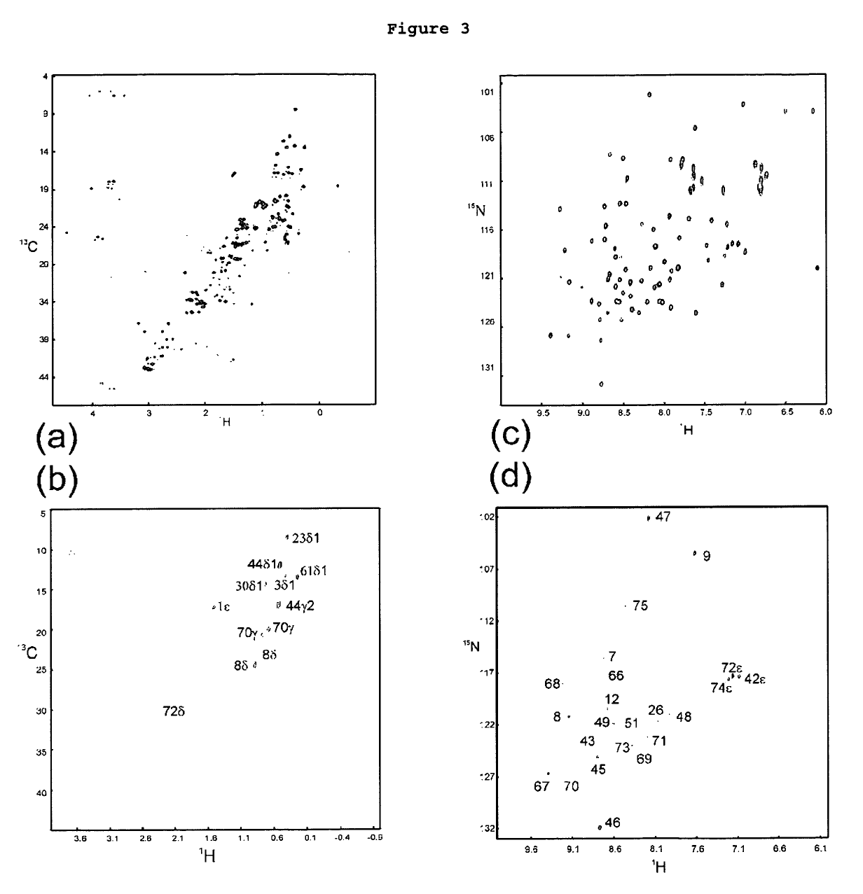

FIG. 3 shows the HSQC spectra of ubiquitin bound to the AUIM peptide.

(a) ¹H{¹³C} HSQC spectrum of REDPRO ubiquitin, a filter was applied to suppress the signal from CH₂D and CH₂ groups.

(b) Difference spectrum between the ¹H{¹³C} HSQC-edited filtered NOESYs obtained after 300 ms and 1 ms mixing time. A threshold of 20% of the maximum signal was applied to discriminate between actual correlations and noise or artifact peaks. No filter was applied so that both signals from CH and CH₂ systems are observed for methyl groups.

(c) ¹H{¹⁵N} HSQC spectrum of ubiquitin.

(d) Difference spectrum between the ¹H{¹⁵N} HSQC-edited filtered NOESYs obtained after 300 ms and 1 ms mixing time. A threshold of 30% of the maximal signal was also applied to identify actual correlations.

All NOESY spectra were obtained with 32 scans.

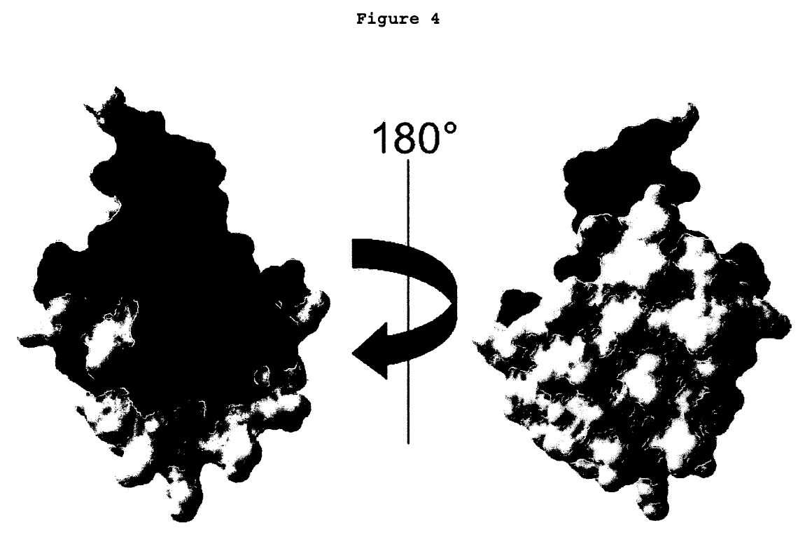

FIG. 4 shows a picture of the interface on ubiquitin obtained from the results of the difference spectra using ¹H {¹⁵N} HSQC edited and ¹H {¹³C} HSQC edited sequences.

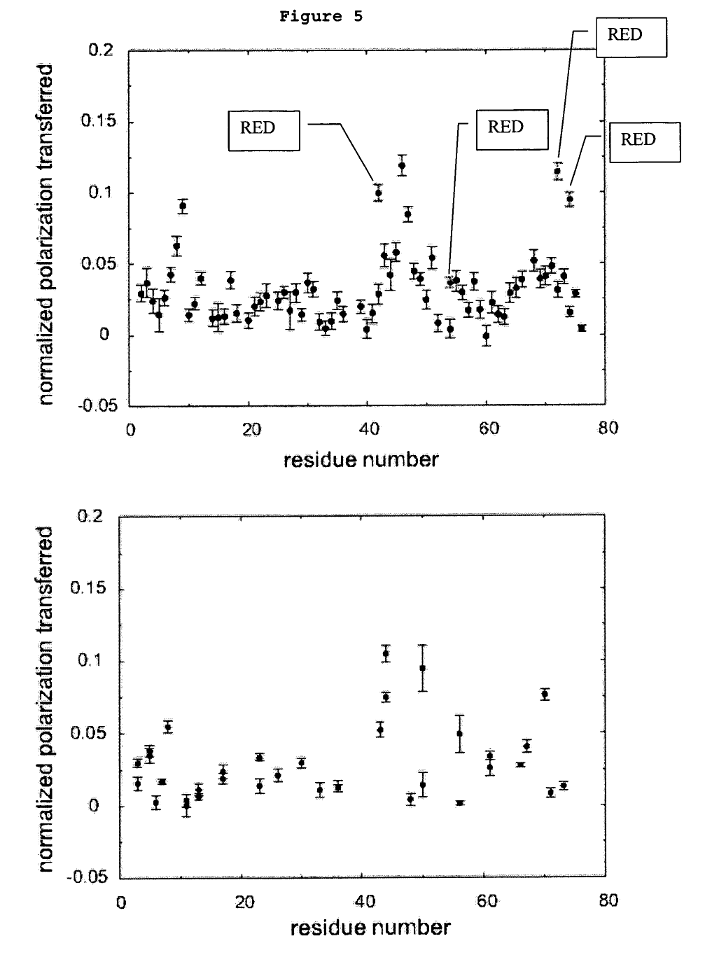

FIG. 5 shows the normalized polarization transferred, (a) for amide protons (black) and arginine side-chains (red), (b) for methyls. A clear difference in the amount of polarization transferred permits the identification of the protons located at the interface. The value obtained can be used as a quantitative information for a subsequent computation of a docking interface. Besides the hydrophobic core of the interface formed by leu 8, ile 44 and val 70 methyls, these data clearly suggest an interaction involving the amide protons of ala 46 and gly 47 as well as the side chains of arg 42, 72 and 74 and the amide of thr 9. Although the amount of polarization transferred is smaller due to fast dynamics, the alternation of high and low transfer efficiency along the tail of ubiquitin (residues 71-76) suggests an orientation of the backbone compatible with the interaction of the side chains from arginine 72 and 74.

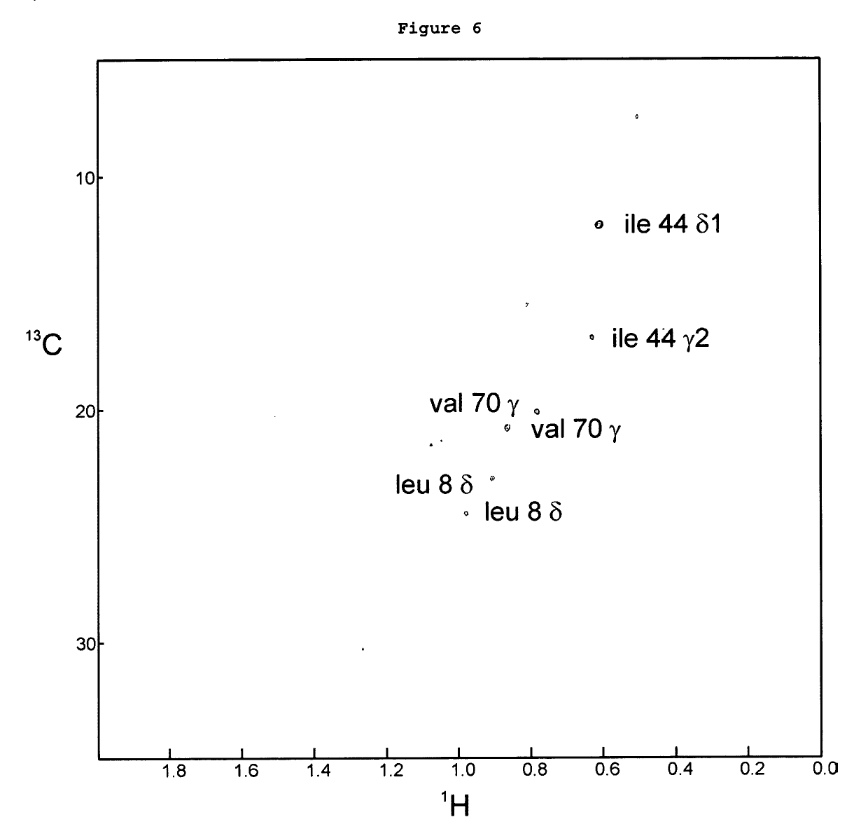

FIG. 6 shows the saturation transfer difference spectrum. To obtain this spectrum, the differences between the saturated and non-saturated spectra obtained with the REDSPRINT sample and the control sample were subtracted. These spectra were obtained on a 700 MHz Bruker Avance spectrometer equipped with a conventional TXI probe.

Each spectrum was acquired with 8 scans.

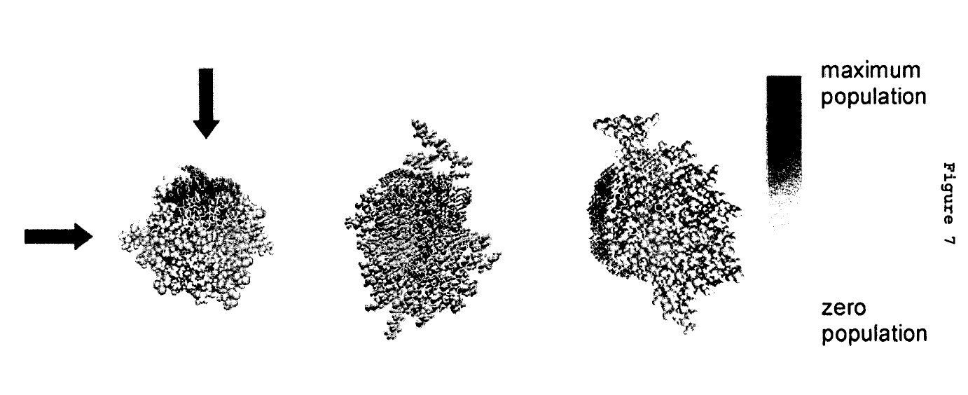

FIG. 7 Three views of ubiquitin and the proton densities for AUIM. Ubiquitin protons with positive constraints are labeled in blue. (a) Arrows indicate the orientations displayed in (b) and (c). The average population of sampled sites is 5% in each tested configuration. Approximately 3000 configurations were averaged to obtain the probabilities displayed. The constraints for protons of the tail of ubiquitin (72-76) have not been included since the structure may be considerably modified by the interaction with the AUIM. One may observe in (a-left) and (c-top) a disjoint region due to the strong constraint linked to the arginine 42 side-chain. In (b-right) and (c-middle) some inconsistent constraints, due either to spin diffusion or polarization transfer from the solvent lead to very small probabilities. Indeed, if a constraint is isolated, the anti-constraints from neighboring protons and van der Waals clashes for buried sites decrease significantly the derived proton population probability.

### DETAILED DESCRIPTION OF THE INVENTION

This invention provides a method for determining the structure or interfacial dynamics of biomolecular complexes, comprising the steps of: (a) providing one or more low proton density target molecule; (b) providing one or more protonated partner molecule that interacts with the target molecule; (c) labeling the target molecule using reduced proton labeling; (d) irridiating the target and partner molecule using ¹H{¹³C} or ¹H{¹⁵N} Heteronuclear Single Quantum Coherence (HSQC)-edited filtered nuclear Overhauser spectroscopy; (e) recording the polarization of the target molecule to obtain spectral data or nuclear Overhauser spectroscopy (nOesy) spectrum of the target molecule; and (f) evaluating the spectral data or nOesy spectrum to obtain the structure or interfacial dynamics of the target and partner molecule. In an embodiment, the structure or interfacial dynamics of the target and partner molecule is obtained by subtracting a NOESY spectrum obtained after zero mixing time to the NOESY spectrum above.

This invention provides a method for determining the structure or interfacial dynamics of biomolecular complexes, comprising the steps of: (a) providing one or more low proton density target molecule; (b) providing one or more protonated partner molecule that interacts with the target molecule; (c) labeling the target molecule using reduced proton labeling; (d) irridiating the target and partner molecule using nuclear Overhauser spectroscopy with an isotopic filter; and (e) recording the polarization of the target molecule to obtain spectral data of the target molecule. In an embodiment, the structure or interfacial dynamics of the target and partner molecule is obtained by normalizing the transferred polarization from the partner molecule to the target molecule employing a spectrum with no isotope filter and with the same mixing time as the spectrum obtained by nuclear Overhauser spectroscopy.

In another embodiment, the transferred polarization is calculated using the formula:
I_{F}(t)/I_{NF}(t)−I_{F}(0)/I_{NF}(0),
Where I is the intensity of a peak, the subscript F and NF refer to filtered and non-filtered experiments and t and 0 are the mixing times.

This invention provides a method for computing the docking surface for the partner molecule onto the target molecule using the normalized transferred polarization. In an embodiment, the docking surface is calculated using the normalized transferred polarization.

In another embodiment, the target molecule or partner molecule described in the above method(s) is a protein, nucleic acid, lipid, carbohydrate, natural or synthetic ligand, intra-protein domain or synthetic molecule. In a further embodiment, the target molecule and partner molecule form a complex comprising two or more biomolecular species of proteins, nucleic acids, carbohydrates, lipids, natural or synthetic ligands or intra-protein domain. In a further embodiment, the target molecule and the partner molecule are copolymers. In a further embodiment, the partner molecule is prepared in a minimum medium with natural isotopes.

In a further embodiment, the ¹⁴N, ¹²C, and ¹H isotopes on the target molecule are replaced selectively by ¹⁵N, ¹³C and ²H isotopes. In a further embodiment, the target molecule is selectively labeled to reduce the ¹H density in a selected spectral region. In a further embodiment, 9% of the hydrogen sites on the target molecule are occupied by ¹H isotopes.

This invention provides a method for determining the structure or interfacial dynamics of biomolecular complexes, wherein the irradiating is performed using selective saturation transfer. In an embodiment, the saturation is achieved after a series of Gaussian-shaped pulses applied with a carrier at 4.3 ppm.

This invention provides an electronic device embodying a computer program to perform the above described method(s) to determine the structure or interfacial dynamics of biomolecular complexes.

This invention provides a computer program for computing the docking surfaces on the target molecule comprising the steps of: (a) defining a three dimensional grid around the target molecule; (b) identifying one or more points around the target molecule wherein polarization transfer was observed; (c) perform Monte Carlo simulation with random population configuration on the grid (The energy function is defined as the sum of the squared deviations from constraints and anti-constraints); and (d) calculating population probabilities from the Monte Carlo results.

This invention provides a method for structure-based drug design, comprising: (a) generating a three dimensional surface of a ligand molecule using the above described methods; (b) performing computer-assisted, structure based drug design with the surface obtained in step (a); and (c) identifying at least one candidate compound that is predicted to have a compatible surface with a target site on the target molecule such that the candidate compound is predicted to bind to the target molecule. In an embodiment, the structure based drug design of step comprises computational screening of one or more databases of chemical compound structures to identify candidate compounds which have structures that are predicted to interact with the three dimensional structure of the target molecule. In another embodiment, the candidate compound having the compound structure identified above is screened or evaluated for biological activity against the target compound.

A new approach to monitor interaction surfaces between molecules, frequently referred to as REDSPRINT (Reduced/standard proton density interface identification), is disclosed. One of the partners (I) is prepared with natural abundance isotopes while the second partner (II) is triply labeled with a reduced proton density [3] (REDPRO). See FIG. 1. Dipolar cross-relaxation from high proton density (I) to the low proton density partner (II) is monitored by filtered NOESY and/or selective saturation transfer experiments. A docking surface is then computed from the constraints collected in all experiments.

The system chosen for this study is the complex between ubiquitin and an ubiquitin interacting motif (UIM) of the protein ataxin [3].

The present invention is further explained by way of the following examples which are to be construed as merely illustrative and not limitative of the remainder of the disclosure in any way whatsoever.

### EXAMPLE 1

Ataxin 3 is a poly- and monoubiquitin binding protein and possesses ubiquitin protease activity. [6] Polyglutamine expansion of Ataxin 3 is implicated in the development of neurodegenerative Machado Joseph disease. Ataxin 3 possesses two ubiquitin interacting motifs (UIMs) mediating its interactions with ubiquitin. UIMs are short 20 amino acid sequences found in many ubiquitin interacting proteins including proteins involved in proteasomal and endocytic degradation pathways. Ataxin 3 UIMs are required for the localization of Ataxin 3 into aggregates in affected neurons and essential for the disease pathology.

1. Principle of the Method

Human ubiquitin (Mw=9.45 kDa) was triply labeled (¹⁵N, ¹³C, ²H) following the REDPRO (reduced proton labeling) method [5] so that, on average, 9% of the hydrogen sites in the protein were occupied by ¹H isotopes (sites are not deuterated uniformly [5]). The ataxin 3 UIM (AUIM), (Mw=5.2 kDa) was prepared in a minimal medium with natural abundance isotopes. For control purposes, a second sample was prepared, for which the AUIM was grown in a D₂O minimal medium. Both samples are prepared in a buffered D₂O:H₂O (80:20) solution. These two systems are represented in FIG. 1.

Cross-relaxation between the high proton density partner towards the REDPRO protein is very efficient whereas cross-relaxation within the REDPRO sample is low, reducing the effects of spin diffusion. Two properties have been exploited to characterize the protons of protein II (ubiquitin) in contact with protein I (ataxin 3 UIM): the differential labeling to carry filtered NOESY experiments as well as the very low alpha proton density in a REDPRO protein to carry out selective saturation transfer methods.

2. Filtered NOESY

In a macromolecule, the longitudinal cross-relaxation through proton-proton dipolar couplings is very efficient. However, the efficiency of a nuclear Overhauser spectroscopy (NOESY) experiment is altered by the very fast longitudinal relaxation of the protons. This is due to the fact that, after a first frequency-labeling period, the longitudinal polarization of the protons is position-dependent within the molecule, so that the very short selective T1 is effective.

In the experiments presented, no frequency labeling is performed before the cross-relaxation period, so that the polarization is not position-dependent and the long nonselective T1 is effective. As a result, efficient polarization transfer occurs from the polarization reservoir (protein I) to the protein II, even in large macromolecular systems. Secondly, in the presence of a very low proton density in protein II, the dipolar cross-relaxation is very inefficient within protein II, so that very little spin-diffusion is expected. Detection of the polarization on protein II is performed through a ¹H{¹³C} HSQC (heteronuclear single quantum correlation) or a ¹H{¹⁵N} HSQC.

The sequence for the ¹H{¹³C} HSQC-edited and ¹H{¹⁵N} HSQC-edited filtered NOESYs are similar to the one developed by Zwahlen et al. [7] All NOESY experiments were run on a Bruker Avance 500 spectrometer equipped with a cold probe.

First, qualitative results can be obtained by subtracting a spectrum obtained after zero mixing time to a filtered NOESY. The latter spectrum contains only the residual polarization that survives after the isotopic filter. The suppression of these artifacts is not perfect due to a dispersion of longitudinal relaxation rates.

Some of the peaks appearing in the difference spectra may come from spin diffusion, as the signals of isoleucine δ1 protons of residues 3, 23, 30 and 61 that are located within the hydrophobic core of ubiquitin. However, these first results, which are straightforward to obtain give a picture of the interface on ubiquitin that compare qualitatively well with one obtained using data acquired with the control sample. See FIG. 4.

A more thorough, quantitative, analysis may be performed to take into account (i) the intrinsic sensitivity of each signal and (ii) the longitudinal relaxation of protons on a site-by-site basis. The latter is difficult to evaluate since the longitudinal relaxation of each proton polarization is multi-exponential. However, it is possible to address these two problems by recording two spectra, with the same mixing times but without the isotopic filter. Then, one can normalize the transferred polarization from the AUIM by the calculation of the quantity:
I_{F}(t)/I_{NF}(t)−I_{F}(0)/I_{NF}(0),
where I is the intensity of a peak, the subscript F and NF refer to filtered and non-filtered experiments and t and 0 are the mixing times. See FIG. 5.
3. Alternative Selective Saturation Transfer Experiment

Although the use of the filtered NOESY experiment with no initial chemical shift labeling should be possible for complexes up to 40-50 kDa, the T2 of protons during the filter—which is 10 ms long—draws a limit for larger systems. Saturation transfer methods do not suffer from such a limitation. In this sample however, the partial protonation of the REDPRO protein prohibits the use of large-band saturation. However, the population of alpha protons is very small in such a sample, often beyond detection. Selective saturation transfer experiments have been designed in which the saturation is achieved after a series of very selective Gaussian or Q3 pulses applied with a carrier at 4.3 ppm. See FIG. 6.

Internal sources of saturation make it difficult to obtain such results without a control sample. However, the design of a lower density REDPRO labeling scheme is expected to improve the results with one REDSPRINT sample.

4. Computation of a Docking Surface on Ubiquitin

The normalized transferred polarization permits to evaluate the sum of the dipolar cross-relaxation rates from the AUIM to an observed proton on ubiquitin. This value can be used to compute a docking surface for the peptide onto ubiquitin.

A program was developed to calculate this surface according to the following process:
(1) A 3D grid is defined around the protein (defined from its Protein Data Bank (pdb) file);
(2) Points are identified within 5 Å of any ubiquitin proton for which a polarization transfer was observed. Those with a non-zero nOe, no steric clash and no cross-relaxation rate higher than any constraint are kept.
(3) A Monte Carlo simulation is carried on with random population configurations on the grid and an energy function defined as the sum of the squared deviations from constraints and anti-constraints (a zero cross-relaxation rate if no data appear in the constraint file).
(4) Population probabilities are calculated from the Monte Carlo results. See FIG. 7.
   5. Conclusion

REDSPRINT is an approach that may be considered as a valuable alternative to saturation transfer for systems up to 50 kDa. Further developments are under process for application to larger size systems. The local and quantitative nature of the information provided makes it suitable for good quality docking of biomolecular complexes. The combination of these data with other quantitative constraints (as orientational constraints [8]) may be an efficient procedure to determine the structure of biomolecular complexes with minimum NMR data.

### EXAMPLE 2

Synthesis of Dilute Isotopes

The host strain of E. coli BL21 is freshly transformed with the expression construct. Cells from overnight culture grown on unlabeled M9 minimal medium in ¹H₂O are collected by centrifugation, washed in phosphate-buffered saline, resuspended in labeling minimal medium, and grown at 37.° C. from OD₆₀₀ (optical density at a 600 nm wavelength) 0.5 to 0.8. In about two to three hours cells adapt to growth in D₂O and reach the indicated cell density. Protein overexpression is induced by addition of 0.5 mM IPTG and the cells are aerated for 20 h at 37° C. Finally, the cells were collected for further purification. The yield of protein using the reduced proton (REDPRO) labeling scheme is similar to that of the standard [U-¹³C, ¹⁵N] labeling scheme.

### EXAMPLE 3

Direct Use of the Physical Principle of the Different Properties of the Reduced Density Material Compared to Standard Density Material

A reduced proton density in any molecule leads to less efficient proton-proton dipolar relaxation. This has two consequences: first, due to longer transverse relaxation times, coherence transfers and detection are more efficient. Secondly, longitudinal cross-relaxation is less efficient. This is of particular interest to detect the interface with any high-proton density system. The high-proton density system behaves as a polarization bath which is only coupled to the few protons from the low-proton density partner located at the interface. Transient (see example 1, point 2) or steady-state (see example 1, point 3) nuclear Overhauser effects can be detected this way.

This principle is general and can be applied to a full range of interfacial systems, far beyond the protein-protein model presented in example 1. This is of use for any kind of complex between two or more biomolecular species as proteins, nucleic acids, carbohydrates, lipids, natural or synthetic ligands. Synthetic molecules can also be studied this way, to obtain information about complexes or the structure of copolymers. Solvation studies can also be performed between a low-proton density solute and a protonated solvent. The properties of the interface between different phases can also be explored with such methods, particularly coupled to diffusion or imaging techniques.

### EXAMPLE 4

Mapping of Side Chain Interactions

The ¹H{¹³C} HSQC-edited filtered NOESY experiment can be carried on to detect contacts between the high-proton density partner and the aliphatic and aromatic protons of the target. Such a method is particularly suited for the detection of hydrophobic contacts.

This experiment was employed to study the interaction between ubiquitin side-chains and the AUIM peptide. The normalized polarization transfer after 300 ms is shown in FIG. 5.b. for methyl groups. The methyls of Leucine 8, Isoleucine 44 and Valine 70 show higher polarization transfer and permit to identify the hydrophobic patch on the surface of ubiquitin.

### EXAMPLE 5

New Method of Detection Using Filtered Homonuclear nOe

The pulse sequence for the detection of amide protons located at the interface is presented in FIG. 2. This sequence is similar to the one published by Zwahlen et al. [7], yet the main difference is that no chemical shift evolution is performed before the mixing time, allowing a longer memory for the spin system during this mixing time. All narrow and wide rectangles are π/2 and π pulses respectively. Carbon-13 frequency shaped pulses are tₚₐ=2.75 ms (a) and t_{pb}=1.793 ms (b) WURST adiabatic pulses [11]. These pulses present an additional phase-modulation so that their effective frequency is at 0 ppm after half their duration. Delays are: τₐ=2.2 ms, τ_{b}=2 ms and τ=(4J_{NH})⁻¹=2.7 ms where J_{NH} is the NH scalar-coupling constant. The additional τ_{c} delay is set so that: 2*τₐ+2*τ_{b}+2*τ_{c}−tₚₐ−t_{pb}=(2J_{NH})⁻¹. The τₘ delay is the cross-relaxation mixing time. The carbon carrier is positioned at 110 ppm for the initial purging pulse as well as for the decoupling pulse during the t₁ evolution, it was set at 27 ppm at every other time. The nitrogen carrier is set at 117 ppm. Composite π pulses compensate for off-resonance effects. During the filter, gradients are G₂+G₄=G₃. For echo-antiecho phase selection, gradient ratios are G₄/G₈=9.9. Sensitivity-enhancement was performed with a PEP scheme (ref). The phase cycle was: φ₁=8{x,x, −x,−x}; φ₂=4{4{y},4{−y}}; φ₃=2{8{y},8{−y}}; φ₄=16{x},16{−x}; φ₅=16{x,−x} and φ_{acq}=8{x,−x,−x,x}. The phase cycle of one pulse before the mixing time is necessary to prevent the build-up of a steady state.

### EXAMPLE 6

Direct Calculation of Docking Surface

The normalized transferred polarization permits to evaluate the sum of the dipolar cross-relaxation rates from the protonated partner to an observed proton on the low-proton density target. This value can be used to compute a docking surface for the first partner onto the target.

The normalized transferred polarization permits an evaluation of the sum of the dipolar cross-relaxation rates from the protonated partner on a ligand to an observed proton on the low-proton density target. A series of factors are neglected to perform a semi-quantitative analysis that does not require too large a set of experiments to record. The site-to-site variation of the transverse relaxation rates of the high proton density ligand and initial polarizations were not taken into account. It is to note that in a large molecule, where longitudinal relaxation is dominated by dipolar cross-relaxation, these site-to-site variations tend to average out. However, this is a less rigorous approximation in a middle-sized system, as the system under study (AUIM, ubiquitin). Nevertheless, one should also notice that an error of a factor of 2 in the evaluated transferred polarization results in only a 12% error in the distance of the cloud.

The Probability cloud can be estimated using the positive constraints of the observed cross relaxation rates, and the negative (‘anti’) constraints of absence of cross relaxation. A constraint file contains in a first column the atom number in the pdb file of the structure of the target. The second column contains the sum of dipolar cross-relaxation rates towards the corresponding observed nucleus. The third column contains an index number that describes the group of equivalent nuclei to which this nucleus pertains; this is particularly designed for methyl groups. A fourth column may contain comments to describe the atoms in a more intuitive manner.

The space in which the calculations are performed is defined as follows. The protein data bank (pdb) coordinate file of the target, including hydrogen positions, is a first input. The origin of the frame is moved either to the center of mass, or to the median point of the protein. A three-dimensional grid is defined, the edges of which are located at least 5 Å from any hydrogen in the target. The resolution of the space is set to 1 Å. With high quality data, a higher resolution can be defined, but one should notice that the number of points considered grows with the inverse cube of the resolution. The next steps of the computation consist in building the portion of the space around the target for which some proton probabilities will be derived. A first test is run to keep only those points with a distance to any constraint less than 5 Å. For these points only, a second test verifies if it lies outside of the protein: the distance with all the proteins atoms from a subset within 7 Å of any constraint is calculated and should be greater than the sum of the van der Waals radii.

Before a last test is run, a table of the dipolar cross-relaxation rates from each point to each group defined in the constraints file is computed. The fluctuations of each proton-proton distance make the real spectral density function difficult to evaluate. A Lipari-Szabo form of the spectral density function is employed so that high frequency contributions are not overly underestimated. Methyl groups are treated as one entity: the cross-relaxation rate calculated is the average between the three proton positions provided in the pdb file. The last test consists in excluding a point for which, for any constraint, a dipolar cross-relaxation rate higher than the constraint was computed.

A final table is calculated. Each line corresponds to a point in the grid that was selected after the three previous tests. The columns correspond to its coordinates as well as its nOe to each proton from the target located within 5 Å of any constraint. This table is the input to a Monte Carlo simulation: a series of random binary distributions of populations in the cloud is generated. The average population of each configuration is set between 5 and 10% of the sites, although a more sophisticated estimate could be made given the typical density of hydrogen in the likely ligand. For each configuration, the sum of the dipolar cross-relaxation rates towards the protons of the target is computed and an energy function E is derived:
E=E_{c}+E_{ac}, [1]
with the energy from measured constraints E_{c}:

$${E}_{c} = {\sum }_{i} {( \frac{{σ}_{tot}^{exp} - {σ}_{tot}^{calc}}{{σ}_{tot}^{exp}} )}_{i}^{2} , \qquad [ 2 ]$$

and the energy from anti-constraints (corresponding to protons for which no cross-relaxation was observed) E_{noc}:

$${E}_{ac} = {\sum }_{j} {( \frac{{σ}_{tot}^{calc}}{{σ}_{tot}^{max}} )}_{j}^{2} . \qquad [ 3 ]$$

where σₜₒₜ^{exp} and σₜₒₜ^{calc} are respectively the measured and calculated sum of all cross-relaxation rates from the protonated partner to the target. σₜₒₜ^{max} is defined as the maximum value of the ensemble of the σₜₒₜ^{exp} values.

A set of configurations (between 500 and a few thousands and from 500000 to a few million tests) is retained. Population probabilities are then derived for each site from a Boltzmann weighted sum of the populations from the selected configurations.

The three tests are run in the sequence presented so that the minimum amount of calculations has to be performed. In the case of the ubiquitin-AUIM complex, the complete selection and computation of the table of dipolar cross-relaxation rates takes two minutes on a dual processor (Pentium IV 1.8 GHz, 512 MB of RAM) PC. The ambiguity of the origin of the polarization transferred towards the target makes it difficult to carry a site-by-site evaluation of the population probability. Therefore, a Monte Carlo approach was chosen for the derivation of population probabilities. About a million configurations can be tested in an overnight calculation with the above-mentioned PC.

This program was experienced to be reasonably resistant to a limited amount of inaccurate constraints. For target protons buried into the target, the steric exclusion test is the main control mechanism. Anti-constraints are important during the Monte Carlo simulation. Indeed, if a “parasitic” constraint (an outlier) is isolated, the sum of neighboring anti-constraints will make the population of surrounding sites unlikely.

### REFERENCES

1. 1 K. J. Walters, A. E. Ferentz, B. J. Hare, P. Hidalgo, A. Jasanoff, H. Matsuo and G. Wagner, Method. Enzymol. 2001, 339, 238.
2. T. Nakanishi, M. Miyazawa, M. Sakakura, H. Tersawa, H. Takahashi and I. Shimada, J. Mol. Biol. 2002, 318, 245.
3. M. P. Foster, D. S. Wuttke, K. R. Clemens, W. Jahnke, I. Rahakrishnan, L. Tennant, M. Reymond, J. Chung and P. E. Wright, J. Biomol. NMR 1998, 12, 51.
4. C. Dominguez, R. Boelens and A. M. J. J. Bonvin, J. Am. Chem. Soc. 2003, 125, 1731.
5. A. Shekhtman, R. Ghose, M. Goger and D. Cowburn, FEBS Lett. 2002, 524, 177.
6. B. Burnett, F. Li and R. N. Pittman, Hum. Mol. Genet. 2003, 12, 3195.
7. C. Zwahlen, P. Legault, S. J. F. Vincent, J. Greenblatt, R. Konrat and L. E. Kay, J. Am. Chem. Soc. 1997, 119, 6711.
8. D. Fushman, R. Varadan, M. Assfalg and O. Walker, Prog. Nucl. Magn. Reson. Spectrosc. 2004, 44, 189.
9. J. Camarero, A. Shektman, E. A. Campbell, M. Chlenov, T. M. Gruber, D. A. Bryant, S. A. Darst, D. Cowburn and T. W. Muir, Proc. Natl. Acad. Sci. 2002, 13, 8536.
10. R. Xu, B. Ayers, D. Cowburn and T. W. Muir, Proc. Natl. Acad. Sci. 1999, 96, 338.
11. E. Kupce and R. Freeman, J. Magn. Reson. A 1995, 115, 273

1. A method for determining the structure or interfacial dynamics of biomolecular complexes, comprising the steps of:
a. providing one or more low proton density target molecule;
b. providing one or more protonated partner molecule that interacts with the target molecule;
c. labeling the target molecule using reduced proton labeling;
d. irridiating the target and partner molecule using ¹H{¹³C} or ¹H{¹⁵N} Heteronuclear Single Quantum Coherence (HSQC)-edited filtered nuclear Overhauser spectroscopy;
e. recording the polarization of the target molecule to obtain nuclear Overhauser spectroscopy (nOess) spectrum of the target molecule; and
f. evaluating the nOesy spectrum to obtain the structure or interfacial dynamics of the target and partner molecule.

2. The method of claim 1, wherein the structure or interfacial dynamics of the target and partner molecule of step (f) is obtained by subtracting a nuclear Overhauser spectroscopy (NOESY) spectrum obtained after zero mixing time to the NOESY spectrum of claim 1.

3. A method for determining the structure or interfacial dynamics of biomolecular complexes, comprising the steps of:
a. providing one or more low proton density target molecule;
b. providing one or more protonated partner molecule that interacts with the target molecule;
c. labeling the target molecule using reduced proton labeling;
d. irridiating the target and partner molecule using nuclear Overhauser spectroscopy without any isotopic filter; and
e. recording the polarization of the target molecule to obtain spectral data of the target molecule.

4. The method of claim 3, wherein the structure or interfacial dynamics of the target and partner molecule is obtained by normalizing the transferred polarization from the partner molecule to the target molecule employing a spectrum with the same mixing time as the spectrum obtained by nuclear Overhauser spectroscopy.

5. The method of claim 2, wherein the transferred polarization is calculated using the formula:
I_{F}(t)/I_{NF}(t)−I_{F}(0)/I_{NF}(0),
Wherein I is the intensity of a peak, the subscript F and NF refer to filtered and non-filtered experiments and t and 0 are the mixing times.

6. (canceled)

7. The method of claim 1, wherein the target molecule or partner molecule is a protein, nucleic acid, lipid, carbohydrate, natural or synthetic ligand, intra-protein domain or synthetic molecule.

8. (canceled)

9. (canceled)

10. The method of claim 1, wherein the target molecule and partner molecule form a complex comprising two or more biomolecular species of proteins, nucleic acids, carbohydrates, lipids, natural or synthetic ligands or intra-protein domain.

11. (canceled)

12. (canceled)

13. The method of claim 1, wherein the target molecule and the partner molecule are copolymers.

14. (canceled)

15. (canceled)

16. The method of claim 1, wherein the partner molecule is prepared in a minimum medium with natural isotopes.

17. (canceled)

18. (canceled)

19. The method of claim 1, wherein the ¹⁴N, ¹²C, and ¹H isotopes on the target molecule are replaced selectively by ¹⁵N, ¹³C and ²H isotopes.

20. (canceled)

21. (canceled)

22. The method of claim 1, wherein the isotopes of the target molecule are selectively labeled to reduce the ¹H density in a selected spectral region.

23. (canceled)

24. (canceled)

25. The method of claim 1, wherein 9% of the hydrogen sites on the target molecule are occupied by ¹H isotopes.

26. (canceled)

27. (canceled)

28. The method of claim 1, the irradiating step (d) is performed using selective saturation transfer.

29. The method of claim 28, wherein the saturation is achieved after a series of Gaussian-shaped pulses applied with a carrier at 4.3 ppm.

30. An electronic device embodying a computer program to perform the method of claim 1 to determine the structure or interfacial dynamics of biomolecular complexes.

31. (canceled)

32. The computer program of claim 30, comprising the steps of:
a. defining a three dimensional grid around the target molecule;
b. identifying one or more points around the target molecule wherein polarization transfer was observed; and
c. perform Monte Carlo simulation with random population configuration on the grid, wherein the energy function is defined as the sum of the squared deviations from constraints and anti-constraints; and
d. calculating population probabilities from the Monte Carlo results.

33. (canceled)

34. A method for structure-based drug design, comprising:
a. generating a three dimensional surface of a ligand molecule using method of claim 1;
b. performing computer-assisted, structure based drug design with the surface obtained in step (a); and
c. identifying at least one candidate compound that is predicted to have a compatible conformation with a target site on the target molecule such that the candidate compound is predicted to bind to the target molecule.

35. (canceled)

36. The method of claim 34, wherein the structure based drug design of step (b) comprises computational screening of one or more databases of chemical compound structures to identify candidate compounds which have structures that are predicted to interact with the three dimensional structure of the target molecule.

37. (canceled)

38. The method of claim 34, further comprising a step of detecting whether the candidate compound having the compound structure identified in (c) has a biological activity.

39. (canceled)

40. (canceled)

41. (canceled)

42. The method of claim 1, wherein the dynamic range or sensitivity of the difference spectra step(s) is/are increased by reducing the density of residual ¹H-¹³C in the ligand by labeling the ligand with ¹²C materials in which ¹³C is depleted.
